(19) United States
(12) Patent Application Publication
(10) Pub. No.: US 2013/0262345 A1
(43) Pub. Date: Oct. 3, 2013

(54) PERSONALIZED DISPENSER SYSTEM

(71) Applicant: GOJO INDUSTRIES, INC., Akron, OH (US)

(72) Inventors: Nick E. Ciavarella, Seven Hills, OH (US); Matthew J. Archer, Aurora, OH (US); Todd Cartner, Uniontown, OH (US); Diane Hillman, North Canton, OH (US); Amanda Jo Copeland, Seville, OH (US)

(21) Appl. No.: 13/826,637

(22) Filed: Mar. 14, 2013

Related U.S. Application Data
(60) Provisional application No. 61/615,937, filed on Mar. 27, 2012.

(51) Int. Cl. A61L 2/00

(52) U.S. Cl. CPC A61L 2/0088
USPC 705/500; 222/183

(57) ABSTRACT

A hand sanitizer dispensing system includes a support case, a container, and a fabric sleeve. The support case may be adapted to be secured to an air vent in an automobile, and includes a body defining an inner volume for receipt of the container. The container includes a product reservoir and a pump in communication with the product reservoir. The fabric sleeve is adapted to be received on the container to allow for personalization of the dispensing system, and the support case may be transparent so that the fabric sleeve is visible. In certain embodiments, the container may be provided with a cap, and pumps including plastic components with infused fragrances may be provided separately for attachment to the container upon removal of the cap.

### CROSS REFERENCE TO RELATED FILINGS

The present invention claims the benefit of the filing of U.S. Provisional Patent Application No. 61/615,937, filed Mar. 27, 2012, incorporated herein by reference.

### FIELD OF THE INVENTION

The system and methods of this disclosure relate to a customizable fluid product dispensing system. More particularly, the system and methods of this disclosure relate to customizable fluid product dispenser system mountable in an automobile.

### BACKGROUND OF THE INVENTION

Personal, portable dispensers for various liquid products are generally known, and are commercially available in a variety of forms. In some of the simplest forms, portable dispensers are provided as containers that can be selectively opened or closed to dispense the liquid product therein. In some embodiments, these containers are pressure actuated, allowing their interior volume to be temporarily decreased in order to dispense some of the liquid product contained therein. These types of containers are very popular for carrying hand sanitizer, hand cleaner, and hand lotion. However, opening and closing of the container typically requires use of two hands.

Some personal dispensers are of a desk top variety. They are somewhat portable in that they are small enough for transporting, but they sit on a counter/desktop for use and are not typically carried around. These dispensers usually include a positive displacement pump for dispensing the product. These pumps may include a piston head that is pushed to dispense liquid product from the container. The pumps typically serve to dispense a unit dose of liquid product upon activation of their dispensing mechanisms thereby providing a consistent volume of product. However, it can sometimes be difficult to dispense the product using the provided pump in circumstances where a consumer does not have both hands readily available.

Dispensers such as those discussed above, are generally manufactured and sold in generic bottles or containers without extensive design or aesthetic features. Adding personalized aesthetic features to these dispensers creates a number of manufacturing and inventory issues that makes the practice unattractive to the manufacturers. For example, where the product to be dispensed has a limited shelf life, the manufacture of several different “designs” of a container may result in increased wastes. However, the ability to customize or personalize consumer products has become increasingly popular and desirable in recent years. Consumers have shown an interest in the ability to create products that reflect their personal interests and tastes.

Thus, there is a need for a hand sanitizer dispensing system that alleviates one or more of the deficiencies of the prior art discussed above.

### SUMMARY OF THE INVENTION

In a first embodiment, the present invention provides a personalized hand sanitizer dispensing system comprising a support case defining an inner volume and including a mounting structure to attach the dispensing system to the inside of a car; a container selectively received in the inner volume of said support case, the container including a pump and a product reservoir containing a fluid product.

In a second embodiment, the present invention provides a dispensing system as in the first embodiment, further comprising a fabric sleeve positioned over the container, the fabric sleeve including a graphic printed thereon.

In a third embodiment, the present invention provides a dispensing system as in either the first or second embodiments, wherein the support case includes a transparent body portion such that at least a portion of the fabric sleeve is viewed through said transparent body.

In a fourth embodiment, the present invention provides a dispensing system as in any of the first through third embodiments, wherein said support case includes a base portion and a lid portion that is pivotable relative to the base portion between an open position and a closed position in order to selectively receive said container.

In a fifth embodiment, the present invention provides a dispensing system as in any of the first through fourth embodiments, wherein at least one of said support case, container, and fabric sleeve is formed from materials that carry a fragrance.

In a sixth embodiment, the present invention provides a dispensing system as in any of the first through fifth embodiments, wherein said mounting structure secures the support case to an air vent in an automobile.

In a seventh embodiment, the present invention provides a dispensing system as in any of the first through sixth embodiments, wherein said pump is accessible outside of said container.

In an eighth embodiment, the present invention provides a dispensing system as in any of the first through seventh embodiments, wherein the pump is a dome pump and is adapted to be actuated by the user placing a hand below an outlet of the pump, with fingers behind the dome pump and a thumb on the dome thereof, depressing the dome with the thumb to dispense product onto the hand below the outlet.

In a ninth embodiment, the present invention provides a dispensing system as in any of the first through eighth embodiments, wherein the pump includes at least one plastic component that carries a substance for imparting a property selected from a fragrance, a color, a moisturizer, a sunscreen and an insect repellant to the fluid product being dispensed.

In a tenth embodiment, the present invention provides a method of merchandising a personalized hand sanitizer dispensing system comprising the steps of offering a support case for sale, the support case including a transparent body defining an inner volume; offering a container for sale, the container selectively received in the inner volume of the transparent body and including a pump and a product reservoir containing a fluid product; and offering a plurality of fabric sleeves for sale, each of the fabric sleeves having a different graphic printed thereon and adapted to be positioned on the container.

In an eleventh embodiment, the present invention provides a method as in the tenth embodiment, wherein the support case includes a mounting mechanism for securing the support case to another object.

In a twelfth embodiment, the present invention provides a method as in either the tenth or eleventh embodiments, wherein the support case includes a mounting mechanism for securing the support case to an air vent in an automobile.

In a thirteenth embodiment, the present invention provides a method as in any of the tenth through twelfth embodiments, wherein the container includes a dome pump.

In a fourteenth embodiment, the present invention provides a method as in any of the tenth through thirteenth embodiments, wherein the body of the support case includes a base portion and a lid portion that is pivotable relative to the base portion between an open position and a closed position in order to selectively receive said container.

In a fifteenth embodiment, the present invention provides a method of merchandising a hand sanitizer dispensing system comprising the steps of offering a support case for sale, the support case including a transparent body defining an inner volume; offering a container for sale, the container adapted to be received in the inner volume of the transparent body and including a removable cap and a product reservoir containing a fluid product; offering a plurality of fabric sleeves for sale, each of the fabric sleeves having a different graphic printed thereon and adapted to be positioned on the container; and offering a plurality of pumps for sale, each of the pumps including plastic components carrying a material for imparting a property selected from fragrance, moisturizer, sunscreen and insect repellant to the fluid product being dispensed, the pumps being adapted to be received on the container to communicate with the product reservoir upon removal of said cap.

In a sixteenth embodiment, the present invention provides a method as in the fifteenth embodiment, wherein said property is a fragrance, and at least one of said fragrances of said pumps is associated with at least one of said graphics of said fabric sleeves.

In a seventeenth embodiment, the present invention provides a method as in either the fifteenth or sixteenth embodiments, wherein said support case includes a mounting mechanism for securing the support case to another object.

In an eighteenth embodiment, the present invention provides a method as in any of the fifteenth through seventeenth embodiments, wherein the said support case includes a mounting mechanism for securing the support case to an air vent in an automobile.

In a nineteenth embodiment, the present invention provides a method as in any of the fifteenth through eighteenth embodiments, wherein the pump is a dome pump.

### BRIEF DESCRIPTION OF THE DRAWINGS

For a full understanding of the invention reference should be made to the following detailed description and the accompanying drawings, wherein:

FIG. 1 is a perspective view of a portable fluid product dispensing system according to the concepts of the present disclosure.

FIG. 2 is a side view of a container including a product reservoir and a pump according to the concepts of the present disclosure.

FIG. 3 is an exploded view of the portable fluid product dispensing system of FIG. 1.

FIG. 4 is a perspective view of the fabric sleeve of the fluid product dispensing system of FIG. 1.

FIG. 5 is a perspective view of another embodiment of a portable fluid product dispensing system according to the concepts of the present disclosure.

### DETAILED DESCRIPTION OF THE ILLUSTRATIVE EMBODIMENTS

In one or more embodiments, the dispensing system of this disclosure may dispense hand sanitizer, hand cleaner, or hand lotion. In certain embodiments, the dispensing system may include a support case adapted to be secured to an air vent of a car, and a container that contains the fluid to be dispensed, the container being received in the support case. In certain embodiments, the support case may be made of a transparent material and a fabric sleeve may be provided around the container, the fabric sleeve being visible through the support case to allow for personalization of the dispensing system. In one or more embodiments, a pump may be provided to interact with the contents of the container and may be formed of a plastic that includes a substance to impart a property to the fluid to be dispensed, thus adding additional personalization options to the dispensing system. In particular embodiments, the property is a fragrance, the pump being made of a fragrance infused plastics. In other embodiments, one or more of the support case or container or sleeve are formed of a fragrance infused plastic.

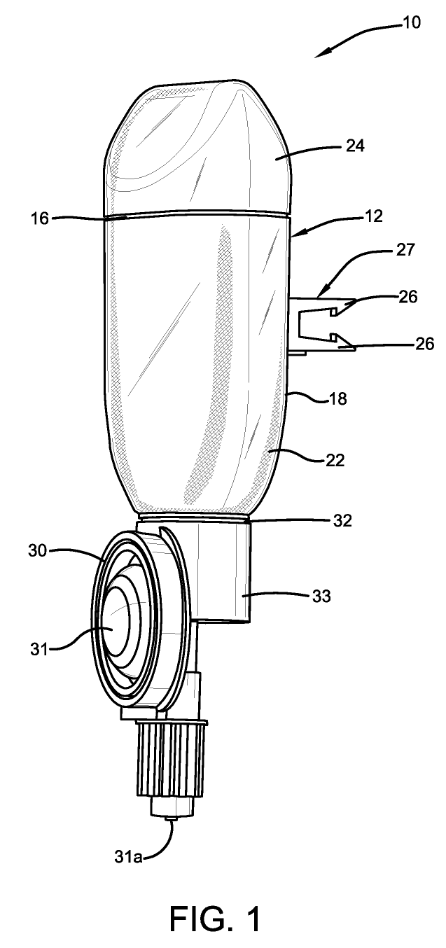

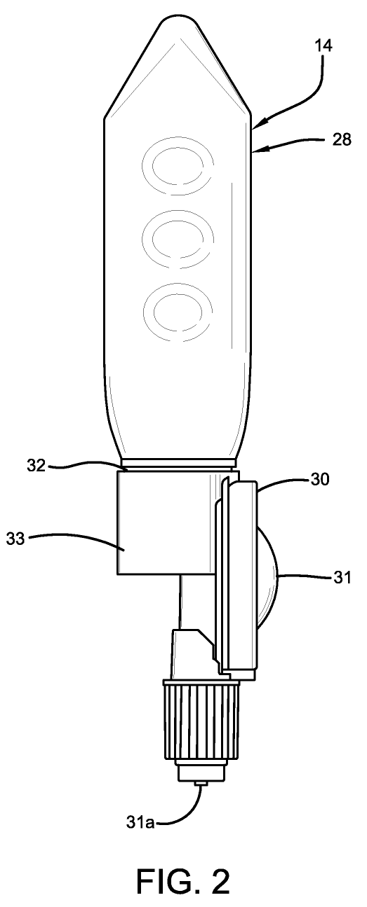

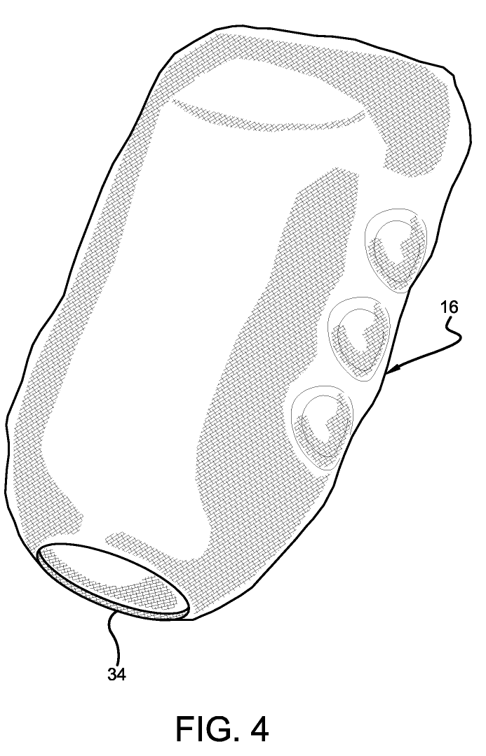

Referring now to FIGS. 1-4, a dispensing system is shown, and is generally indicated by the numeral 10. Dispensing system 10 is adapted to be mounted in an automobile, but it will be appreciated by those skilled in the art that the system may be configured for use in other environments. Dispensing system 10 includes a support case 12, a container 14 that contains a fluid product to be dispensed and is adapted to be received in the support case 12, and a fabric sleeve 16 positioned around the container 14 to permit customizing of the dispensing system 10.

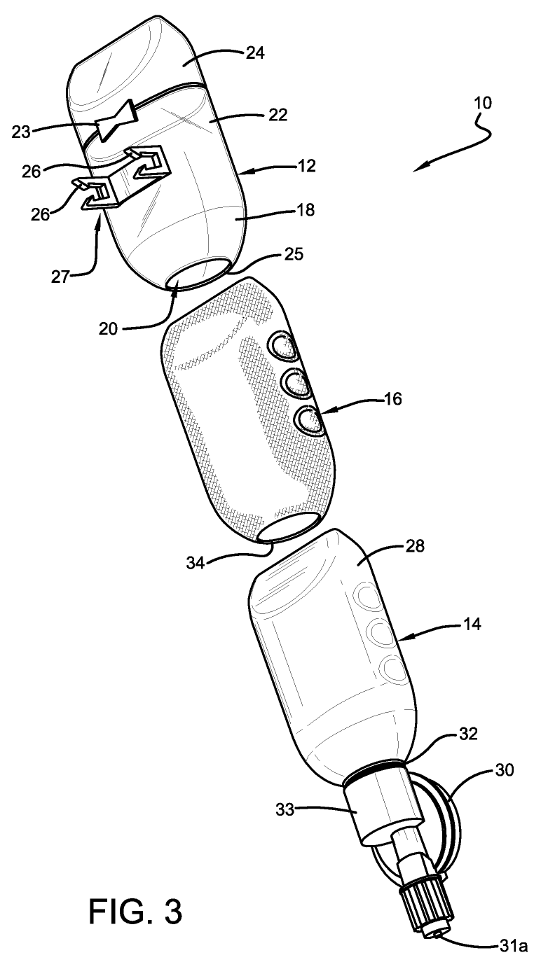

In one or more embodiments, the support case 12 may include a body 18 defining an inner cavity 20. The body 18 may include a base portion 22 and a lid portion 24 pivoting relative to the base portion 22 to permit opening and closing of the support case. The lid portion 24 may be pivotally secured to the base portion 22 by any suitable method or mechanism known to those skilled in the art. In a particular embodiment, and as shown in FIG. 3, the lid portion 24 is secured to the base portion 22 by a living hinge 23, which is a part of the body 18. Alternatively, a separate hinge mechanism may be secured to the body 18 to hingedly connect the lid portion 24 and base portion 22. A latch mechanism (not shown) may also be provided to secure the lid portion 24 in a closed position. The body 18 and inner cavity 20 may be provided in any desired shape and size adapted to receive a correspondingly shaped and sized container therein. In one or more embodiments, the body 18 may include an opening 25 to allow a portion of the container 14 to project from the inner cavity 20 of the support case, as will be discussed in more detail below.

In one or more embodiments, the body 18 of the support case 12 may be formed from a transparent material. In the same or other embodiments, the body 18 of the support case 12 may be formed from a plastic material. In certain embodiments, the body 18 of the support case 12 may be formed from a transparent plastic material, such as, for example, acrylic materials.

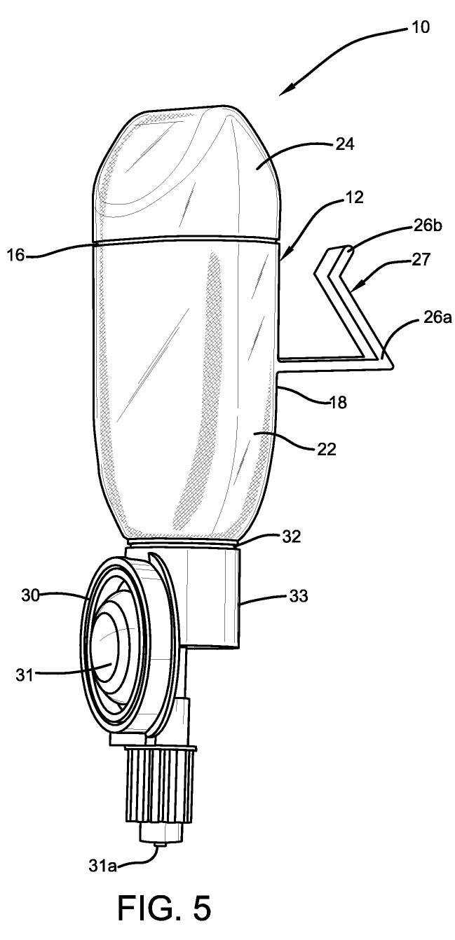

In certain embodiments, the support case 12 may also include a mounting structure 27. In the embodiment shown in FIG. 3, the mounting structure 27 includes one or more legs 26 extending from the backside of the support case 12. The legs 26 may be adapted to secure the body 18 to an object or surface. In one or more embodiments, the legs 26 are adapted to secure the body 18 to the louvers of an air vent within an automobile. In another embodiment, as shown in FIG. 5, the mounting structure 27 may include a leg 26a that is flexible and resilient, and includes an angled end portion 26b. The leg 26a is adapted to secure the support case 12 to a sun visor or a seat back storage pocket of an automobile. In still other embodiments, the mounting structure 27 may be adapted to secure the support case 12 in or to a cup holder of a vehicle. While several particular mounting structures 27 are shown, it is contemplated that any suitable mounting structure may be used to mount the support case 12 to an air vent, to a cup holder, to a sun visor, and to a seat back storage pocket in an automobile, or to any other desired location or structure. In particular embodiments, the mounting structure 27 is specifically designed to interact with the air vents of a car such that the mounted dispensing system 10 would be provided at the air vent.

The container 14 may be of any known type or configuration for supplying fluids or gels for personal use. These might include hand sanitizer, hand cleaner, hand lotion, bug repellant, sunscreens, and the like. In certain embodiments, the container 14 may include a product reservoir 28 containing the fluid product to be dispensed and a pump 30 that is actuated to cause dispensing of the fluid product. The pump 30 may project from or otherwise be accessible through the opening 25 in the body 18 to allow actuation of the dispensing system.

In one or more embodiments, the pump 30 may be a dome pump that, when the flexible dome member 31 is pressed, causes displacement of a dose of fluid product from the pump outlet 31a, and when released creates a vacuum to draw another dose of the fluid product into the pump 30 from the product reservoir 28. Such pumps are generally known to those skilled in the art. Suitable dome pump technology for fluid dispensers is disclosed in U.S. Pat. Nos. 6,216,916 and 7,891,583, which are incorporated herein by reference for that purpose. It is also contemplated that other known types of pumps may be utilized with the container 14 to dispense the fluid product. The container 14 is standard, or universal, so that the same container may be used with any dispensing system 10 along with other components that provide personalization and are adapted to work with the container, as will be discussed in more detail below.

In a particular embodiment such as that shown in FIG. 1, the mounting structure 27 is used to mount the dispensing system to the air vent of a car, with the dome pump 31 at a bottom most position with the outlet 31a thereof pointing downwardly such that a user can reach over to the vent, place fingers behind the dome pump and a thumb on the dome thereof, depressing the dome with the thumb to dispense product onto the fingers or hand that would be below the outlet. This is a user-friendly configuration suitable for use in a car.

In one or more embodiments, the universal container 14 may include a threaded neck 32 that, when manufactured, receives a threaded cap (not shown) thereon. A pump 30, including a threaded collar 33, may be purchased by a consumer for attachment to the threaded neck 32 upon removal of the cap. In certain embodiments, a fragrance may be infused into one or more of the plastic components of the pump 30. A variety of pumps having differing fragrances may be made available for purchase, each being adapted to communicate with the product reservoir 28 of the container 14.

The specific fragrances infused in the plastic pump components may be any suitable fragrances that consumers may find appealing. Reference is made to U.S. Patent Publication 2004/0028779, which discloses a method of infusing plastic with a fragrance, and is incorporated herein by reference for that purpose. Reference is also made to U.S. Pat. No. 7,005,152, wherein a plastic component of a bottle includes fragranced oils to emit a perceivable fragrance. Notably, in the aforementioned publication and patent, the scented plastic serves merely to directly cause the user to perceive a scent directly from the scented plastic. In distinction, the present invention, the scented plastic components of the pump are infused with a fragrance in order to transfer the selected fragrance to the fluid product (e.g. hand sanitizer) being dispensed, such that the fragrance is then physically transferred to the user upon use of the fluid product.

It is also contemplated that the plastic components of the pump that come into contact with the liquid being dispensed may be made of a plastic material that carries other materials and/or properties, as may be apparent to those skilled in the art. The plastic components may be infused with or coated with a substance that is adapted to impart the desired properties or additives to the fluid product being dispensed. For example, in certain embodiments, the pump components may be coated or infused with a substance that imparts a color to the liquid being dispensed. In other embodiments, the plastic pump components may be coated or infused with a substance that imparts a moisturizer to the liquid being dispensed. Similarly, the plastic pump components may be coated or infused with a substance that imparts sunscreen or insect repellants to the liquid being dispensed.

In yet other embodiments, the fabric sleeve 16 is formed form materials that carry a fragrance. In yet other embodiments, the support case 12 is formed from materials that carry a fragrance. In yet other embodiments, the container 14 is formed from materials that carry a fragrance. Notably, in embodiments where the mounting structure 27 is specifically designed to interact with the air vents of a car such that the mounted dispensing system 10 would be provided at the air vent, forming one or more of the fabric sleeve 16 or support case 12 or container 14 from materials that carry a fragrance would provide the added benefit of achieving an air-freshening effect inside the car, particularly when the air vent is on and blowing air over the dispensing system 10.

In one or more embodiments, the fabric sleeve 16 may be adapted to be received on the container 14. In certain embodiments, the fabric sleeve 16 may cover substantially the entire outer surface of the container 14. In other embodiments, the fabric sleeve 16 may cover only a portion of the outer surface of the container 14. In one or more embodiments, the fabric sleeve 16 may include an opening 34 that aligns with the opening 25 in the support case 12 when the system 10 is assembled. The fabric sleeve may be made of any suitable fabric material. In one or more embodiments, the fabric sleeve 16 may possess a sufficient degree of elasticity to allow it to be stretched and positioned on or around the container 14.

In one or more embodiments, the fabric sleeve 16 may include graphics or a pattern printed on at least one surface to provide personalization of the dispensing system 10. In certain embodiments, the graphics or patterns may be provided only on an exterior surface of the fabric sleeve 16. In other embodiments, the fabric sleeve 16 may be reversible and include graphics on both surfaces, and the graphics or patterns on the opposing surfaces of the fabric sleeve 16 may be the same or different. The images, patterns, and colors of the fabric sleeve are limitless, and may include any patterns or graphics that appeal to consumers. For example, flower, fruit, or camouflage patterns may be printed on the fabric sleeve 16. Alternatively, as another example, the fabric sleeve 16 may include abstract designs or graphics.

In one or more embodiments, a consumer may purchase a container 14, and a support case 12. The consumer may then select and purchase a fabric sleeve having graphics or patterns that are appealing to them. A variety of options of fabric sleeves may be presented for sale at the point of purchase. In certain embodiments, the container 14 may be purchased with a pump 30 positioned thereon. In other embodiments, the container 14 may be purchased with a disposable cap thereon, and the pump may be purchased separately.

Optionally, where the container is not provided with a pump, the consumer may select and purchase a pump 30 that is adapted to be secured to the container 14 and that, in some embodiments, includes an infused fragrance. Like the fabric sleeve 16, a variety of options of pumps having different fragrances may be presented for sale at the point of purchase. In certain embodiments, the fragrance of the pump may relate to or may be associated with the graphics on the fabric sleeve. For example, where the fabric sleeve 16 includes a flower image, the fragrance of the pump may be a flower scent. In a particular embodiment, a fabric sleeve 16 and pump 30 having related graphics and scents may be packaged together for sale.

Once all components of the dispensing system 10 are purchased, the consumer may then slide or otherwise position the fabric sleeve 16 onto the container 14 so that the graphics or patterns of the fabric sleeve are visible on an exterior of the container. The container 14, with the fabric sleeve 16, may then be positioned within the body 18 of the support case 12, and the lid portion 24 may be closed to secure the container and fabric sleeve within the support case. In certain embodiments, a portion of the container 14, including the pump 30, may project from the support case 12 to facilitate actuation of the dispensing system 10. The transparent nature of the support case 12 allows the fabric sleeve 16 to be viewed through the support case.

The support case 12 may then be secured to the louvers of an air vent in an automobile or other suitable surface so that the pump 30 is accessible to a driver or passenger in the vehicle. The stable support provided by the support case and the accessible nature of the pump 30, will allow for quick and easy actuation of the pump and dispensing of a product from the dispensing system 10. The universal container 14 may be replaced when empty with another universal container 14, and the support case 12, fabric sleeve 16, and optionally the pump 30 may be reused with a new container 14. Thus, a consumer need only purchase the personalized components of the dispensing system 10 a single time, and future purchases may be limited to replacement containers 14. Alternatively, the design of the dispensing system 10 may be changed at any time by selecting and purchasing a different fabric sleeve and/or pump.

It is thus evident that a dispensing system constructed as described herein substantially improves the art. Only particular embodiment(s) have been presented and described in detail, and the invention should not be limited by the drawings or the description provided. For an appreciation of the true scope and breadth of the invention, reference should be made only to the following claims.

What is claimed is:

1. A personalized hand sanitizer dispensing system comprising:
(a) a support case defining an inner volume and including a mounting structure to attach the dispensing system to the inside of a car;
(b) a container selectively received in the inner volume of said support case, the container including a pump and a product reservoir containing a fluid product.

2. The system of claim 1, further comprising a fabric sleeve positioned over the container, the fabric sleeve including a graphic printed thereon.

3. The system of claim 2, wherein the support case includes a transparent body portion such that at least a portion of the fabric sleeve is viewed through said transparent body.

4. The system of claim 3, wherein said support case includes a base portion and a lid portion that is pivotable relative to the base portion between an open position and a closed position in order to selectively receive said container.

5. The system of claim 2, wherein at least one of said support case, container, and fabric sleeve is formed from a material that carries a fragrance.

6. The system of claim 1, wherein said mounting structure secures the support case to an air vent in an automobile.

7. The system of claim 1, wherein said pump is accessible outside of said container.

8. The system of claim 7, wherein the pump is a dome pump and is adapted to be actuated by the user placing a hand below an outlet of the pump, with fingers behind the dome pump and a thumb on the dome thereof, depressing the dome with the thumb to dispense product onto the hand below the outlet.

9. The system of claim 7, wherein the pump includes at least one plastic component that carries a substance for imparting a property selected from a fragrance, a color, a moisturizer, a sunscreen and an insect repellant to the fluid product being dispensed.

10. A method of merchandising a personalized hand sanitizer dispensing system comprising the steps of:
(a) offering a support case for sale, the support case including a transparent body defining an inner volume;
(b) offering a container for sale, the container selectively received in the inner volume of the transparent body and including a pump and a product reservoir containing a fluid product; and
(c) offering a plurality of fabric sleeves for sale, each of the fabric sleeves having a different graphic printed thereon and adapted to be positioned on the container.

11. The method of claim 10, wherein the support case includes a mounting mechanism for securing the support case to another object.

12. The method of claim 10, wherein the support case includes a mounting mechanism for securing the support case to an air vent in an automobile.

13. The method of claim 10, wherein the container includes a dome pump.

14. The method of claim 13, wherein the body of the support case includes a base portion and a lid portion that is pivotable relative to the base portion between an open position and a closed position in order to selectively receive said container.

15. A method of merchandising a hand sanitizer dispensing system comprising the steps of:
(a) offering a support case for sale, the support case including a transparent body defining an inner volume;
(b) offering a container for sale, the container adapted to be received in the inner volume of the transparent body and including a removable cap and a product reservoir containing a fluid product;
(c) offering a plurality of fabric sleeves for sale, each of the fabric sleeves having a different graphic printed thereon and adapted to be positioned on the container; and
(d) offering a plurality of pumps for sale, each of the pumps including plastic components carrying a material for imparting a property selected from fragrance, moisturizer, sunscreen and insect repellant to the fluid product being dispensed, the pumps being adapted to be received on the container to communicate with the product reservoir upon removal of said cap.

16. The method of claim 15, wherein said property is a fragrance, and at least one of said fragrances of said pumps is associated with at least one of said graphics of said fabric sleeves.

17. The method of claim 15, wherein said support case includes a mounting mechanism for securing the support case to another object.

18. The method of claim 15, wherein the said support case includes a mounting mechanism for securing the support case to an air vent in an automobile.

19. The method of claim 18, wherein the pump is a dome pump.
